(19) United States
(12) Patent Application Publication
(10) Pub. No.: US 2015/0020810 A1
(43) Pub. Date: Jan. 22, 2015

(54) SIMULTANEOUS POSTIVE AND NEGATIVE AIRWAY PRESSURE PUMP

(71) Applicant: Howard D. Stupak, Southport, CT (US)

(72) Inventor: Howard D. Stupak, Southport, CT (US)

(21) Appl. No.: 14/338,105

(22) Filed: Jul. 22, 2014

Related U.S. Application Data
(60) Provisional application No. 61/958,237, filed on Jul. 22, 2013.

(51) Int. Cl. A61M 16/00; A61M 16/04; A61M 16/06

(52) U.S. Cl. CPC A61M 16/009; A61M 16/006; A61M 16/0666; A61M 16/049; A61M 16/0066; A61M 16/06
USPC 128/205.19

(57) ABSTRACT

A system and method of using both positive and negative airflow to create differential pressures in the oral and nasal cavities for support of the pharynx to treat obstructive sleep apnea.

### CROSS-REFERENCE TO RELATED APPLICATIONS

This application claims priority to U.S. Provisional Patent Application No. 61/958,237 entitled “FACIAL-MOUNTED SIMULTANEOUS POSITIVE AND NEGATIVE AIRWAY PRESSURE PUMP,” filed Jul. 22, 2013, the entire disclosure of which is incorporated herein by reference.

### FEDERALLY SPONSORED RESEARCH

None.

### SEQUENCE LISTING

None.

### BACKGROUND

Disclosed herein is a treatment for obstructive sleep apnea that uses both positive and negative air pressure to maintain the airway during sleep.

In patients suffering from obstructive sleep apnea (OSA), the pharynx collapses during sleep, causing obstruction of the upper airway. This obstruction in turn causes interruptions in breathing, a reduction in blood oxygen saturation, and frequent, partial awakenings in order to restore breathing. The interruptions in sleep in turn are associated with daytime sleepiness, restless sleep, headaches, trouble concentrating, mood changes, increased blood pressure, weight gain, and other problems.

Currently, the conventional understanding of obstructive sleep apnea syndrome (OSAS) is that obstructions during OSAS are due to the Bernoulli Effect of a tube, where negative pressure in the pharynx causes increased airspeed, and draws its collapsible walls inward, creating obstruction. This obstruction is conventionally treated with continuous positive pressure delivered by CPAP machine. Those skilled in the art are familiar with the extensive literature describing CPAP and its improvements to masks, pumps, hoses, etc. CPAP essentially delivers positive pressure to the airway to force open a collapsing tongue, soft palate and pharynx during sleep. The standard CPAP therapy of today consists of an electric powered table mounted air pump that can deliver humidified or nonhumidified positive pressure through a long to a variety of masks from a full-face cushioned mask to a specialized nasal pillows mask (U.S. Pat. No. No. 7,578,294 B2). Recently, a wearable CPAP has been disclosed, where a reduced pump size is worn on a sleeve (Patent application US 2007/0163600) as well as a system of CPAP where recycled expired air is used to store energy for a pump (Patent application US 2012/0234323 A1).

CPAP therapy is effective to treat OSAS, but it is unpleasant, and patient compliance with therapy is relatively low. Some studies estimate that between 29% and 83% of patients remove the CPAP early in the night or skip use altogether.

Others (U.S. Patent 8,122,890 B2) have suggested that oral-applied negative pressure in conjunction with a physical appliance which positions the tongue can pull the collapsible tongue and soft palate forward to prevent subsequent collapse into the airway, but teach that negative pressure alone is insufficient to reposition the tongue.

Still needed is a non-surgical treatment for OSA which is effective and more tolerable for patients.

### BRIEF DESCRIPTION OF THE DRAWINGS

Embodiments will be readily understood by the following detailed description in conjunction with the accompanying drawings. To facilitate this description, like reference numerals designate like structural elements. Embodiments are illustrated by way of example and not by way of limitation in the figures of the accompanying drawings.

FIG. 1 is a cut-away view of a face in profile in which the aerodynamic pattern of the simultaneous positive nasal and negative oral air pressure created by a facial mounted pump can be seen.

FIG. 2 is a profile view of a pump in accordance with one embodiment of the invention mounted on a mask structure on a patient's face.

FIG. 3 is a perspective view of a pump in accordance with various aspects of the invention mounted on support struts on the face.

FIG. 4 is a top-down, cross section view of the oral cavity of a patient using one embodiment of the pump disclosed herein.

FIG. 5 is a top-down, cross section view of an alternative embodiment of the system described herein mounted on the chest of a patient.

FIG. 6 is a perspective view of an alternative embodiment of the invention disclosed herein, wherein the diaphragm pump is mounted on the chest and is powered by chest-wall motion.

FIG. 7 is a perspective view of an associated diagnostic anemometer used to measure nasal oral airflow ratio.

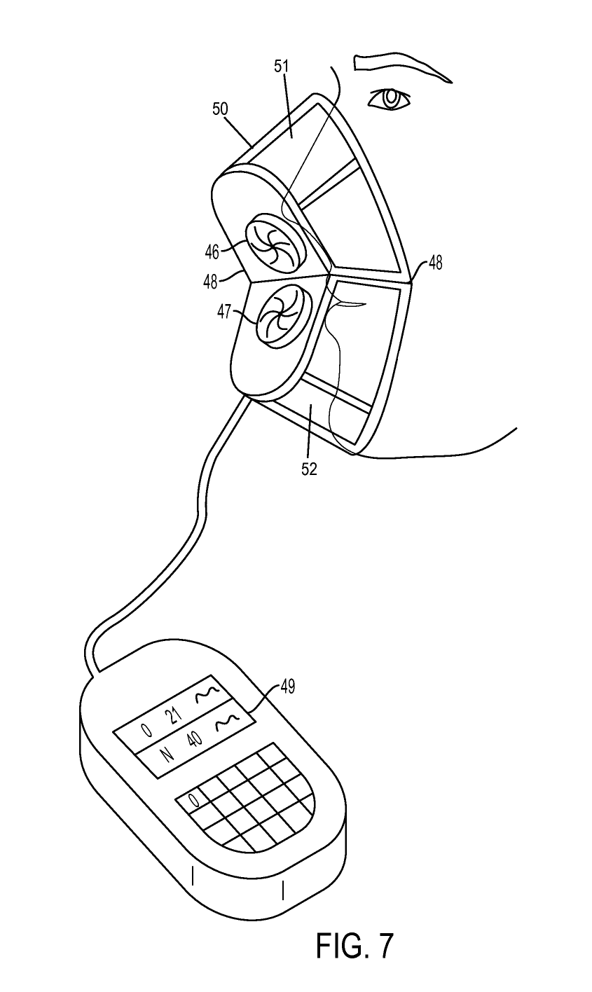

### DETAILED DESCRIPTION

In the following detailed description, reference is made to the accompanying drawings that form a part hereof wherein like numerals designate like parts throughout, and in which is shown by way of illustration embodiments that may be practiced. It is to be understood that other embodiments may be utilized and structural or logical changes may be made without departing from the scope of the present disclosure. Therefore, the following detailed description is not to be taken in a limiting sense.

Various operations may be described as multiple discrete actions or operations in turn, in a manner that is most helpful in understanding the disclosed subject matter. However, the order of description should not be construed as to imply that these operations are necessarily order dependent. In particular, these operations may not be performed in the order of presentation. Operations described may be performed in a different order than the described embodiment. Various additional operations may be performed and/or described operations may be omitted in additional embodiments.

The description uses the phrases “in an embodiment,” or “in embodiments,” which may each refer to one or more of the same or different embodiments. Furthermore, the terms “comprising,” “including,” “having,” and the like, as used with respect to embodiments of the present disclosure, are synonymous.

Disclosed herein are various embodiments of several sleep support systems which employ both positive and negative pressure, and thus support the airway using less airflow than is possible using CPAP therapy.

It is conventionally believed that the external nose and nasal passages do not play a substantial role in OSA. It has been observed that nasal surgery and nasal strips have little effect on the severity of OSA, and has been believed to have minimal value as a treatment for OSA. CPAP therapy delivers a column of positive air pressure indiscriminately through the mouth or nose or both to stent the airway open. The positive air pressure must be sufficient to prevent the airway from collapsing, and so significant airflow is necessary for the therapy to be effective. Whether that airflow is delivered to the airway via the nose or mouth is not conventionally thought to matter.

The inventor has shown in a recent study how it is not just airflow, but a synergistic relationship between positive nasal and negative oral airflow that creates the ideal airway aerodynamics to support the tongue and palate in prevention of collapse. The inventor herein has discovered that the soft palate (or tongue) functions as an “airfoil” with two surfaces exposed to two different air pressures, and thus when viewed in a sagittal plane roughly resemble a cross sectional view of an airplane wing or sail in the pharyngeal space.

An airfoil (or wing) experiences “lift” when it experiences differential air pressure on opposing surfaces and the airfoil moves in the direction of lower pressure. Another example of this is when a door slams shut due to the pressurization of a room by an air conditioner where the adjacent room is not air-conditioned.

In treatment of OSA, while the traditional concept of tube collapse can be useful, it may be the more complex model of the palate and tongue acting as airfoils that is more relevant. Increasing laminal airflow in the nasal cavity creates positive pressure which, in combination with negative pressure in the oral cavity, effectively dilates the pharynx by causing the tongue and soft palate to change positions in response to the air pressure difference like an airfoil would. This repositioning of the tongue and dilation of the pharynx can prevent airway collapse. Positive pressure airflow enters the nose and supports the posterior palate from collapsing in the nasal pharynx, while a negative pressure in the oral cavity draws the palate and tongue forward, and also preventing collapse. Conversely, positive airway pressure in the mouth and lower pressure in the nasal cavity may have the opposite effect.

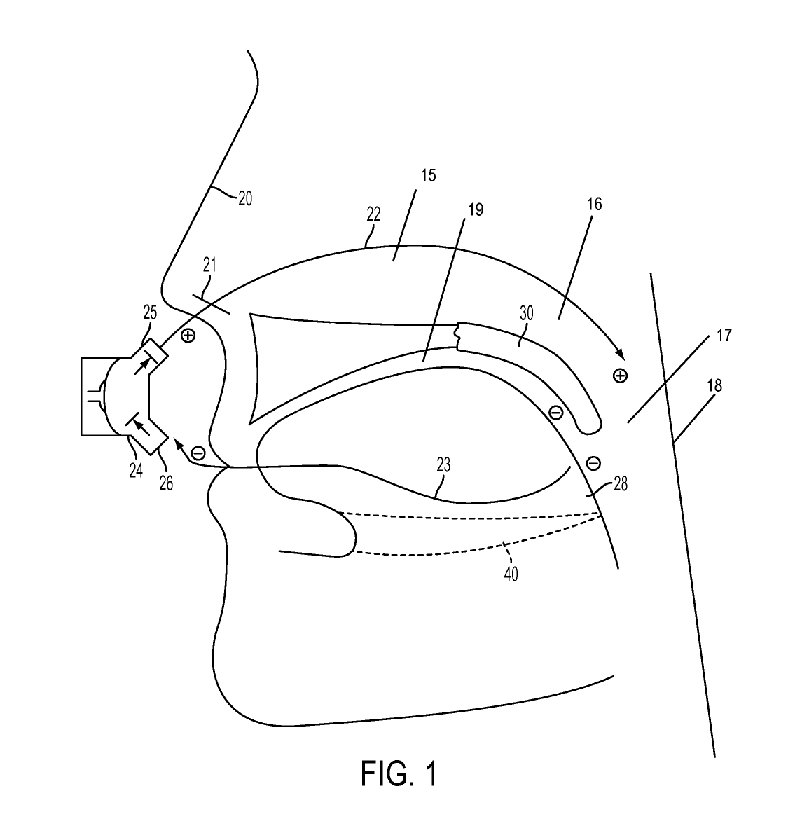

As shown in FIG. 1, in a supine patient gravity exerts posterior displacement of the palate 30 and tongue 28, which is exacerbated by the decreased muscle tone during sleep. When the nasal airflow and pressure is greater than the oral airflow and pressure, the result lift displaces the palate 30 and tongue 28 toward the oral cavity 19 and away from the posterior pharyngeal wall 18, thus dilating the pharyngeal opening 17 and preventing collapse during diminished muscle tone. An optimal approach to the support of the airway during sleep lies in the synergistic effects of both positive and negative air pressure. By leveraging these synergistic effects, support of the airway can be achieved with less positive pressure than would be effective without the concurrent use of negative pressure, and vice-versa.

The various embodiments of the present invention permit the application of both positive and negative airflow, adding positive pressure to the nasal cavity 15 and nasopharynx 16 and thus the pharyngeal side of the palate 30 and tongue 28 while suction is applied to oral cavity 19, thus drawing the tongue 28 and palate 30 forward.

The device includes a pump, optionally a diaphragm or centrifuge pump, which can be mounted on the face between the nose and mouth. The pump may be small enough to be facially mounted because of targeted and increased efficiency of airway support (detailed below) that delivers positive pressure to the nose from the positive airflow side of the pump. The pump may also deliver suction or negative pressure to the oral cavity with the suction side of the pump. The device capitalizes on improved knowledge of pharyngeal aerodynamics to permit airway support more efficiently.

The present device may offer any of several distinct advantages over the prior art. These advantages result from efficiencies in the pump and airway aerodynamics (outlined below) that permit the treatment of OSA with less airflow than is required by CPAP therapy, permitting the patient to be free of long hoses, table-top pumps, and potentially electrical wire sources and also permitting improved portability over conventional CPAP devices.

In some embodiments of the present invention, one single pump provides positive pressure through its outflow port to the nose, and negative pressure or vacuum through its inflow port, resulting in greater efficiency in the engine use. Because positive pressure is provided to the nose simultaneously with negative pressure supporting the mouth or oral airway, less pressure is needed to either treatment, as the combination of treatments is synergistic.

A small pump can be facially mounted, resulting in some embodiments with short hoses from the inflow and outflow valves. Because the hoses from the inflow and outflow valves of the pump may be extremely short (just a few centimeters) compared to desktop mounted pumps, the pressure drop or loss of pressure due to hose length is very minimal for positive and negative pressure, thus greatly enhancing the efficiency of pumping. Since the above mentioned hoses or tubes are traversing such a short distance, they can be precisely angled toward their target, in other words, be mounted directly in the nose and mouth for precision air placement rather than gross airflow through a full-faced mask.

Large quantities of air under pressure are not required to support the pharyngeal structures during sleep, because targeted airflow positive in the nasopharynx supports the palate and tongue from collapsing backward, while targeted negative pressure vacuum airflow in the mouth will pull the tongue and palate forward thus preventing their collapse. Thus, targeted airflow to better understood pharyngeal aerodynamics permits less pump capacity than would mass amounts of gross airflow generally blown into the pharynx, thus requiring smaller pump size.

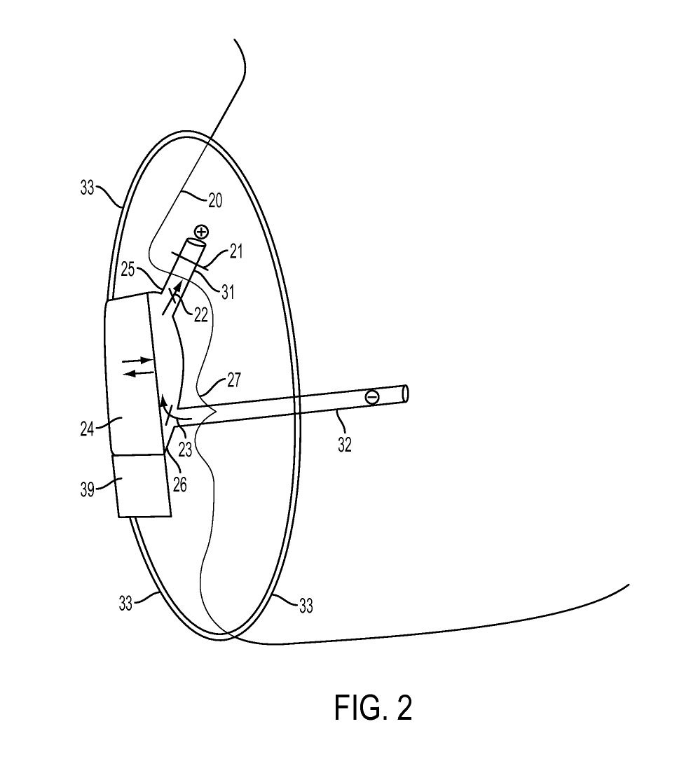

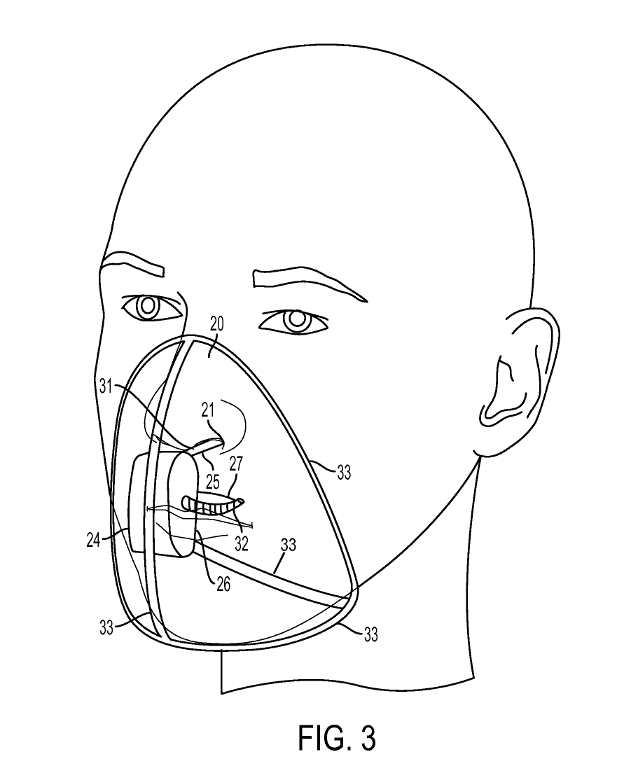

As shown in FIGS. 1-5, the system comprises an air pump 24 which can be facially mounted and which is in communication with a nasal valve 25 and one or more tubes 31 which are configured and positioned to direct air through the nostril 21 to deliver positive airflow pressure (as shown by arrow 22) into the nasal cavity 15 and nasopharynx 16. The pump is in further communication with an oral valve 26 and one or more tubes 32 which are configured and positioned to remove air from the oral cavity 19 (as shown by arrow 23), thus affecting the pharyngeal aerodynamics during sleep to support the tongue and palate from collapsing without requiring high pressures and a large table top pump like a CPAP machine.

In some embodiments of the pump 24, using electromechanical driven pistons, a flexible diaphragm is compressed and relaxed alternatively, creating a diaphragm pumping action on a sealed chamber. Air is pumped out of a one directional outflow valve in a tube 31 entering the nostril 21, aimed specifically toward the nasopharynx 16 in an angle of attack as shown by arrow 22 that will maximize the lift of the palatal and tongue structures. Simultaneously, release of the compressed diaphragm will remove air from the oral cavity via another tube. The resulting vacuum effect in the oral cavity and draw the palate and tongue forward away from the posterior pharyngeal wall. The combined positive and negative airflow is synergistic and will create a cyclical airflow pattern in the pharynx, thus creating favorable lift to the collapsible structures in the pharynx.

As shown in FIGS. 1-3, the pump 24 can be positioned and secured on a patient's face during use via the tubes 31 which enter the nose 20 (one or both nostrils) and the tubes 32 which enter the mouth 27 as well as optional support limbs 33 distributing the force of weight and vibration along the bony structure of the face.

Pressurized air can be delivered to both nostrils, or it can be delivered to one nostril while the other nostril is left unencumbered to inhale ambient, non-pressurized air.

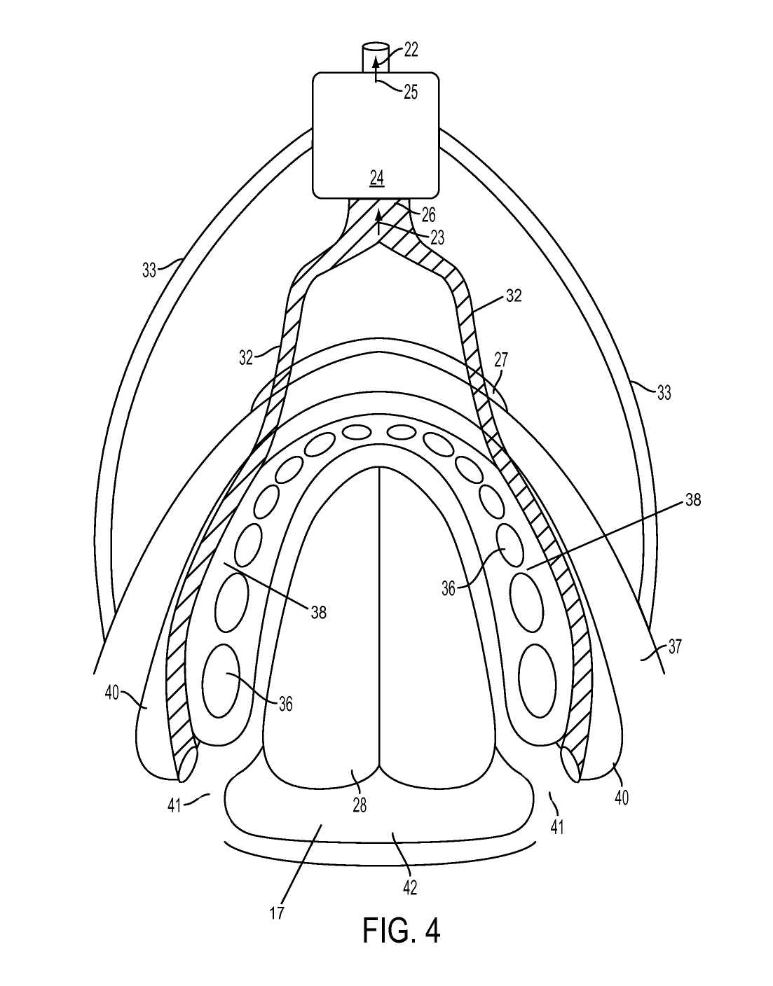

As shown in FIG. 4, in the oral cavity, the vacuum tube 32 from the pump 24 may be connected from the oral valve 26, or pump intake valve, and bifurcated into two branches, each of which may enter the fissures or corners of the mouth. Each of the branches may be placed inside each cheek 37 and along each outer gum-line or alveolar ridge 38 to lead to the retro-molar spaces 41 behind the alveolar ridge, where there is a direct connection from the buccal space to the pharyngeal opening 17 of the pharynx 42. This allows direct negative air pressure to the base of tongue 28 and palate. For purposes of orientation, teeth are indicated by reference number 36.

Alternatively, the lips may be sealed in order to enhance the creation of low air pressure in the oral cavity. Negative air pressure may be delivered through a tube which works in conjunction with a membrane or structure which is substantially impervious to air and which is placed between the patient's lips and incisors in order to substantially seal the oral cavity against any flow of air from outside the patient's mouth. For example, the vacuum tube 24 may extend through the center of a suction-cup like structure. The suction cup-like structure should be formed from a flexible polymer which can conform to the physiology of lips and teeth and which is substantially impervious to air. A vacuum tube extends from the pump through the central region of the structure. The structure can be placed between the patient's lips and the labial surfaces of the patient's incisors such that the vacuum tube extends into the patient's oral cavity. The vacuum tube inserted into a mouth otherwise sealed against the inflow of air may then more efficiently create low air pressure within the oral cavity.

The pump 24 may be powered by an alternating current source or via batteries 39 which can be integral with the pump and mask and may be rechargeable. A ring of a rigid but soft material may surround the nose and chin as the base of the support structure 33, with struts 33 extending to the mounted pump 24. The face mask need not seal to the face and need not be impervious to air, as air pressure from the pump is delivered directly to one or two nostrils via tubes 31. Straps supporting the mask and/or pump may be used as well. Other forms of pumps including centrifugal air pumps can be used as well.

The diaphragm pump may have an optional trigger that links the pumping action with respiration. One link could be an electromyographic electrode, a thin wire attached to the upper chest and neck that reads chest wall motion, and would trigger pumping action. An alternative would be continuous or cycled regular interval pumping action. The suction in the mouth may not be adequate for priming or refilling the positive pressure of the pump, thus a backup inflow nozzle open to fresh air may be required as well.

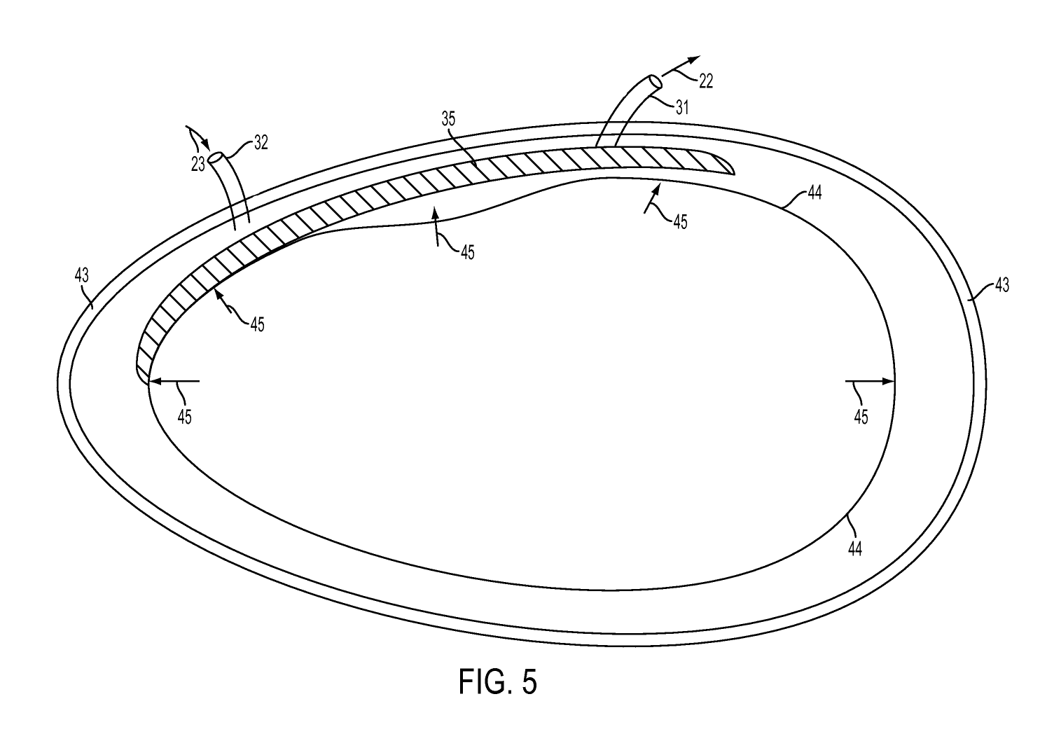

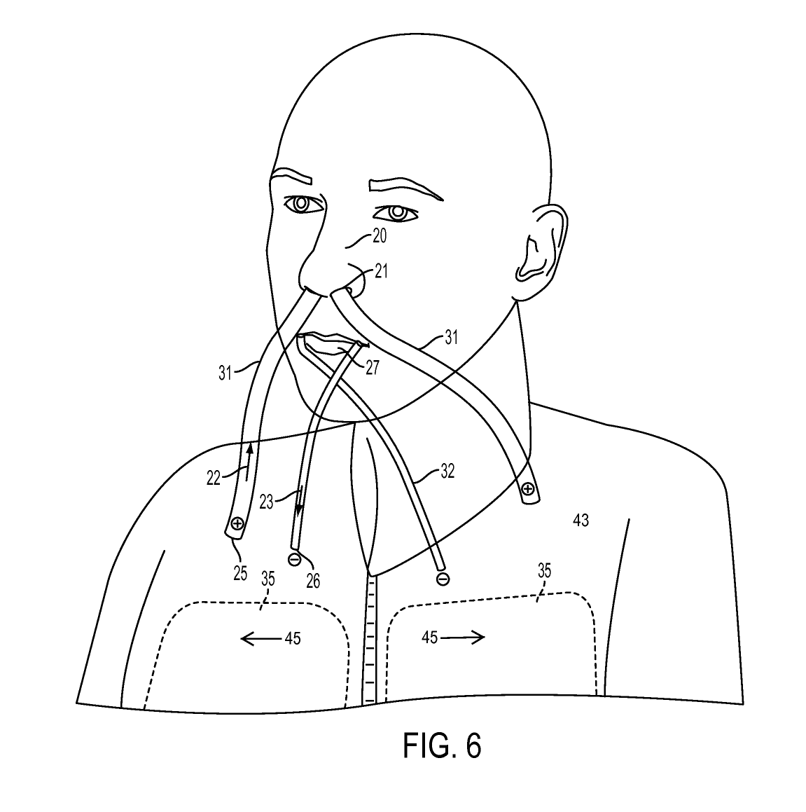

An alternative embodiment involves the use of the same intake and outflow on a diaphragm pump, except instead of an electromechanical piston powering the pump, the pump may be powered by the patient/user's chest wall. As shown in FIGS. 5 and 6, a large resilient diaphragm bladder 35 is mounted on the patient's chest 44 under a rigid corset 43. The corset and diaphragm bladder can be sewn into a vest or shirt type garment. As the chest wall expands upon inspiration, shown by arrows 45, the diaphragm bladder 35 is compressed, and air is expelled outward through the outflow tube 31 connected to the nostril 21 of the nose 20, directing positive pressure and airflow into the nasopharynx. When the chest wall subsequently relaxes, and the diaphragm bladder rebounds and takes in air, thus creating a negative pressure vacuum via tube 32 to the oral cavity (mouth). This creates a similar aerodynamic pattern as the facial mounted pump, but is clearly mounted on the chest. The system requires no additional power source.

Finally, an alternative embodiment to determine appropriateness of this system for patients involves the use of an anemometer with two impellers 46, 47 mounted on a similar facial mask 50, with the digital readout 49 held separately in the palm of the hand. The mask 50 includes a partition 48 between oral 52 and nasal 51 compartments. The ratio of nasal airflow to oral airflow over several minutes while the patient is resting in the supine position is calculated. An elevated reading of supine oral to nasal airflow ratio (SONAR) would indicate good candidacy for the facial mounted nasal oral airflow pump in the treatment of sleep apnea.

The inventor performed a single subject study in which an individual having a baseline Apnea-Hypopnea index of 15 in a supine position, indicating moderate sleep apnea, used a pump to deliver positive pressure to the nasal cavity and negative pressure to the oral cavity, in accordance with some embodiments of the invention described herein, while sleeping in a supine position. While using the pump, the subject's Apnea-Hypopnea index was 1, indicating that the pump had resolved the subject's sleep apnea symptoms.

What is claimed is:

1. A method of treating obstructive sleep apnea comprising:
(a) providing a pump configured to deliver both pressurized air and vacuum;
(b) using said pump to deliver pressurized air to a nasal cavity of a patient; and
(c) using said pump to concurrently remove air from an oral cavity of said patient.

2. The method of claim 1 wherein said pump is mounted on a mask which is adapted to distribute weight to bony structures of a patient's face.

3. The method of claim 1 wherein said pump is configured to deliver pressurized air to said nasal cavity of said patient through a tube inserted into said patient's nostril.

4. The method of claim 1 wherein said pump is configured to remove said air from said oral cavity of said patient through a tube inserted into said patient's mouth.

5. The method of claim 4 wherein said tube is interconnected with a structure comprised of a flexible polymeric material, said structure configured to fit between said patient's lips and labial surface of at least one said patient's incisor, said structure further configured to substantially inhibit the passage of air into said patient's mouth.

6. The method of claim 1 wherein said pump comprises a diaphragm located within a chamber, further wherein said diaphragm is alternatively compressed and relaxed during operation.

7. The method of claim 1 wherein said pump comprises a resilient air bladder adapted to be placed in contact with said patient's chest during use, said bladder further adapted to expel air when said patient inhales and take in air when said patient exhales.

8. The method of claim 4 wherein said tube is bifurcated into two branches, and each said branch is placed along each alveolar ridge within said oral cavity of said patient.
